(19) United States
(12) Patent Application Publication
(10) Pub. No.: US 2011/0091557 A1
(43) Pub. Date: Apr. 21, 2011

(54) COMPOSITE MATERIAL

(75) Inventors: Joanna Jayne Buckley, York (GB); Adam Fraser Lee, York (GB); Karen Wilson, York (GB)

(73) Assignee: Smith & Nephew PLC, London (GB)

(21) Appl. No.: 12/812,676

(22) PCT Filed: Jan. 14, 2009
(86) PCT No.: PCT/GB2009/000085

§ 371 Date: Dec. 2, 2010

(30) Foreign Application Priority Data
Jan. 14, 2008 (GB) 0800549.8

(51) Int. Cl. A61K 33/38; A61K 9/00; A61K 9/14; A61P 17/02; A61P 31/00; B05D 1/12; B82Y 5/00

(52) U.S. Cl. 424/489; 424/618; 424/400; 424/619; 264/234; 427/212; 977/773; 977/915

(57) ABSTRACT

The invention relates to a composite material, a precursor for forming the composite material and a method of forming the composite material from the precursor. The invention also relates to the use of said composite material and in particular to its use as an antibacterial or antimicrobial agent.

The present invention relates to a composite material, a precursor for forming the composite material and a method of forming the composite material from the precursor. The invention also relates to the use of said composite material and in particular to its use as an antibacterial or antimicrobial agent.

Antimicrobial agents are regularly used in the medical field for imparting sterility to prevent infection. Silver, one of the most widely used antimicrobial agents, has a pronounced ability to kill a broad spectrum of infectious bacteria. Since silver's first clinical application as an antimicrobial agent, the form in which it is applied has diversified. For example, catheters can be coated with elemental silver to help to reduce biofilm formation, dilute silver nitrate solution is used to treat eye infections, and silver sulfadiazine is employed in the treatment of general infections.

Silver present in antimicrobial agents, will commonly be in the form of a salt of the silver. The rate of release of the silver from the salt, and the nature of the silver species liberated, is critical to the efficacy of the antimicrobial agent. Generally, silver salts dissolve readily in water and thus also in wound fluid. Due to the generally high solubility of silver salts, the silver is released rapidly from the antimicrobial agent and thus consumed quickly requiring frequent addition of fresh antimicrobial agent. In the area of wound care, for example, this would require regular changes in a patients wound dressing or bandage which may not only be impractical and time consuming but also potentially harmful for the patient, delaying recovery time.

It is an object of the present invention to provide an antimicrobial agent or composite material having antimicrobial properties which will release a potent form of silver more consistently over a prolonged period of time. It is also an object of the present invention to provide a precursor for forming the composite material and a method of forming the composite material from the precursor.

Therefore, according to a first aspect of the present invention, there is provided a composite material having antimicrobial properties comprising a substrate and a silver salt.

The substrate can be amorphous or porous. Preferably, the substrate is porous. The porosity of the substrate increases the surface area which provides for a greater surface area per unit volume. This allows for a greater amount of the salt to be added to the substrate and for the silver salt to be more finely distributed over its surface. Both of these conditions provide release of more of the silver from the salt than would otherwise be released from the salt itself. These conditions also enable the selective formation of a particular chemical form of silver salt.

Preferably, the substrate is mesoporous, having a pore size of between 1 nm and 50 nm. More preferably, the pore size is between 2 nm and 15 nm. Generally, the size of the salt particles is influenced by the amount or concentration of the silver salt on the substrate. The greater the amount or concentration of the salt the greater will be the particle size. Therefore, the lower the concentration of silver salt, providing a smaller size particle of salt which is more finely dispersed over the substrate, the faster the silver is released from the salt. The chemical nature of the salt is independent of the concentration.

The substrate generally comprises a metal or metal oxide. Preferably, the substrate is aluminium or aluminium oxide.

Preferably, the salt is silver carbonate (Ag₂CO₃). The silver carbonate is ideally in the form of nanoparticles effectively forming a layer on the surface of the substrate. The salt in nanoparticle form releases more silver than the salt in bulk or macroparticle form. It has also be found that the combined effect of the layer of salt in nanoparticle form on a substrate, in particular of aluminium oxide, provides for a more controlled release of silver from the salt than would occur from the same salt in bulk form.

Where the composite material is used in medical applications, the silver salt is preferably water soluable. Ideally, the salt is also biocompatible and thermally degradeable.

The composite material can be formed by the physical treatment of a composite material precursor or by the chemical and physical treatment of a pre-synthesised substrate.

Therefore, according to a second aspect of the present invention, there is provided a composite material precursor comprising, by weight percent, an aluminium-alkoxide (5-50%), a silver salt (5-50%), a surfactant (0-25%), an alcohol (0-70%) and a solvent (0-25%).

Preferably, the aluminium-alkoxide is aluminium-sec-butoxide and the silver salt is silver nitrate. The surfactant may be any suitable type of surfactant which is capable of templating the aluminium-alkoxide and silver salt to form a solution. For example, the surfactant can be lauric acid. Preferably, the solvent is polar covalent and is ideally water.

According to a third aspect of the present invention, there is provided a method of forming the composite material from the precursor comprising the steps of:

mixing, by weight percent, an aluminium-alkoxide (5-50%), a silver salt (5-50%), a surfactant (0-25%), an alcohol (0-70%) and water (0-25%) to form a solution;

heating the solution; and

allowing the solution to cool to form a solid composite material having a modified silver salt deposited on its surface.

Preferably, the solution is heated to at least 500° C. On heating the solution, the mixture reacts to form aluminium oxide which on cooling forms the substrate. The surfactant not only improves the solubility of the aluminium-alkoxide and silver salt in solution, but also controls the pore size and thus porosity of the substrate formed.

Preferably, the surfactant is present in solution, by weight percent, in the range 5-15%. The surfactant may be, for example, lauric acid.

Preferably, the aluminium-alkoxide is Al-sec-butoxide and is present in solution, by weight percent, in the range of 10-40%.

Preferably, the silver salt in solution is silver nitrate and is present, by weight percent, in the range of 10-40%.

Ideally, the solution is exposed to ambient air or to an atmosphere which containes CO₂. As the solution cools in air, the silver ions at the surface of the forming substrate react with the CO₂ and are modified to form nanoparticles of silver carbonate.

Preferably, the size of the silver salt nanoparticles are in the range of 1 nm to 9 nm, more preferably, in the range of 3 nm to 6 nm.

it has been found that the substrate in the form of a mesoporous substrate and in particular an aluminium oxide mesoporous substrate dramatically increases the affinity of silver ions for CO₂, substantially aiding modification and transformation of the silver salt to a stable carbonate salt. The mesoporous substrate and in particular the aluminium oxide mesoporous substrate promotes formation of the salt into nanoparticles at the surface of the substrate.

It has been found that silver salt in nanoparticle form releases far more silver than the salt in macroparticle or bulk form. The combination of the mesoporous substrate and in particular an aluminium oxide mesoporous substrate and the silver salt, in particular the silver carbonate, releases silver more consistently over a more prolonged period of time than the salt itself in bulk form.

Instead of forming the composite material from a single solution, the substrate of the composite material can be formed first with the silver salt applied at a later stage.

Therefore, according to a fourth aspect of the present invention, there is provided a method of forming the composite material comprising the steps of:

providing a substrate;

coating the surface of the substrate with a layer comprising silver salt;

heating the substrate and layer of silver salt; and

allowing the substrate and layer of silver salt to cool to form a solid composite material having a modified silver salt deposited on the surface of the substrate.

The substrate may typically be formed by mixing, by weight percent, an aluminium-alkoxide (5-99%), a surfactant (1-25%), alcohol (0-20%) and water (0-25%) to form a solution and heating the solution to at least 500° C. to form a solid porous substrate.

Subsequently, and advantageously after the substrate has cooled to approximately room temperature, the surface of the substrate is coated with a layer comprising silver salt, preferably silver nitrate, and heated to at least 500° C. In a similar fashion to the third aspect, the silver salt at the surface of the substrate on cooling reacts with the CO₂ and is modified to form nanoparticles of silver carbonate.

The invention may be carried into practice in various ways, and embodiments thereof will now be described by way of example only with reference to the accompanying drawings in which:

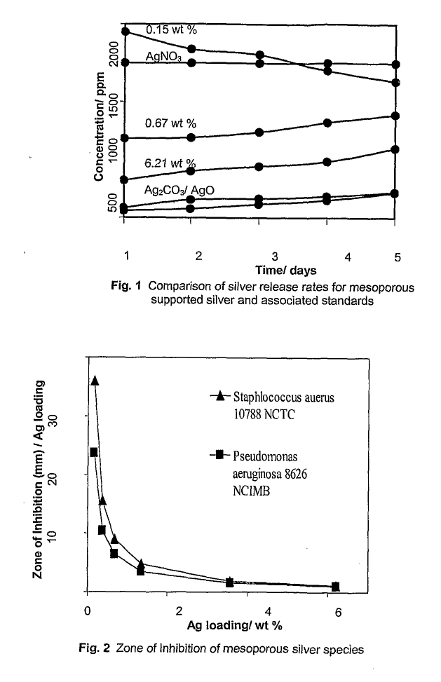

FIG. 1 shows the rate of release of silver from various composite materials according to the present invention having a mesoporous substrate;

FIG. 2 compares composite materials of the present invention, having different surface areas and concentrations of the substrate and silver salt respectively, demonstrating that the combination of low concentrations of silver salt with high surface area substrates provide a composite material with better antimicrobial properties than the combination of higher concentrations of silver salt with lower surface area substrates;

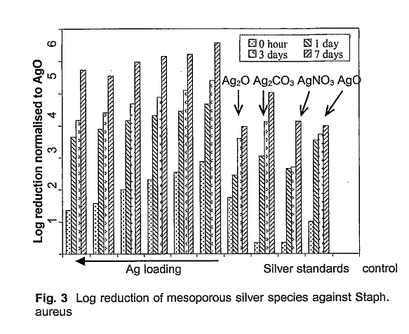

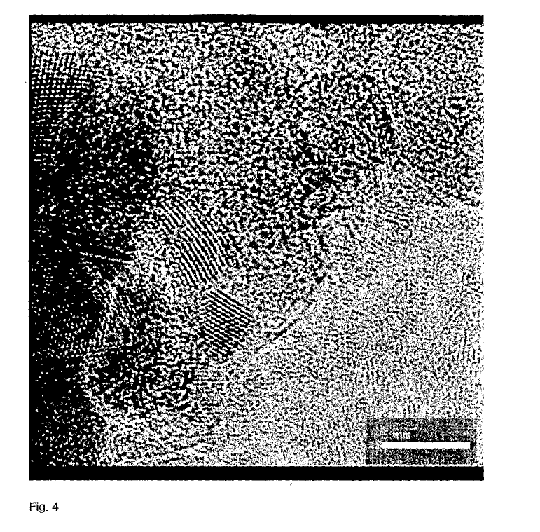

FIG. 3 demonstrates the efficacy of various composites according to the present invention as antimicrobial agents against the staphylococus bacteria; and

FIG. 4 visualises the antibacterial silver carbonate nanoparticles on a mesoporous alumina substrate prepared according to the fourth aspect of this invention.

A method of how to form a composite material according to the present invention will hereinafter be described.

To form the substrate, an aluminium hydroxide suspension is formed by hydrolysis of 43.8 g of aluminium tri-sec-butoxide with 10.3 g of deionised water and 275 g of 1-propanol. After stirring the hydroxide suspension for one hour at room temperature, 10.8 g of lauric acid is added to form a mixture. The mixture is aged for 24 hours at room temperature and then heated under static conditions at 110° C. for 2 days. The solid aluminium oxide formed is filtered, washed with ethanol and dried at room temperature to form aluminium oxide powder. The aluminium oxide powder is calcined in air at 500° C. for 3 hours with a ramp rate of 20° C. per minute to form a solid mesoporous aluminium oxide substrate.

Using the incipient wetness technique, a silver salt coating is prepared by mixing portions of the prepared aluminium oxide powder with water and silver nitrate to form a slurry. A layer of the slurry is coated onto the surface of the solid mesoporous aluminium oxide substrate. The substrate with coating is heated at 100° C. to remove the water from the slurry. After removal of all or most of the water from the slurry, the substrate and coating are calcined at 500° C. in air for 3 hours with a ramp rate of 20° C. per minute. After calcination and on cooling to room temperature, the silver incorporated within the substrate reacts with the CO₂ in the air forming silver carbonate nanoparticles to create a composite material having a solid mesoporous aluminium oxide substrate with an intergral surface layer comprising silver carbonate nanoparticles, a portion of the integral surface layer impregnating the surface of the substrate.

Referring now to the drawings and initially to FIG. 1, there is shown comparative data for the release of silver from composite material samples using a silver selective electrode. The composite material samples comprise a mesoporous aluminium oxide substrate with various silver salts and concentrations thereof, ranging from 200 to 2000 ppm. FIG. 1 demonstrates that there is a good correlation between the quantity of silver salt present in the composite and the release rate of silver therefrom. The smaller the quantity of silver salt present in the composite, the faster the silver is released. This is believed to be due to the smaller particle size and greater dispersity of salt which can be achieved in the composite when smaller quantities of salt are used.

FIG. 1 also demonstrates that all of the composite samples tested, when normalised, release proportionally more silver initially and over the 5 days than the equivalent bulk silver salts, some of which contain as much as ˜80 wt % silver.

Composite samples containing nanoparticles of Ag₂CO₃ of various concentrations were tested for their ability to inhibit bacterial growth against two strains of organisms, Staphylococus aureus 10788 NCTC and Pseudomonas aeruginosa 8626 NCIMB. Staphylococus aureus 10788 NCTC and Pseudomonas aeruginosa 8626 NCIMB are examples of Gram-positive and Gram-negative bacteria respectively, typically found on the skin.

The results obtained, when normalised to the quantity of silver salt present in each composite, demonstrate that composites having lower concentrations of silver salt and higher surface areas present better antibacterial characteristics than composites having a higher concentration of silver salt with lower surface areas. This is quite clearly shown in FIG. 2.

Bacteria inhibition rates give a good indication as an initial microbial test. However, kill kinetics are more important in wound management. Therefore, to more fully test the composite samples containing nanoparticles of Ag₂CO₃, a full kill test (log reduction) was performed over 7 days with the bacteria, Gram-negative Staphylococcus aureus 10788 NCTC. This organism was chosen as it is very difficult to kill.

As can be seen clearly from FIG. 3 all composites exibit far superior antibacterial properties in comparison to their bulk counterpart silver salts. To be classed as bactericidal, industry standards require antibacterial agents to provide a three fold log (99.9%) reduction in the number of viable bacteria. All the sample composites surpass this requirement by several orders of magnitude. The equivalent bulk silver salts, struggle to reach this requirement. However, it was found that the sample composite containing silver carbonate as the salt surpasses this requirement within 24 hours. It is also found that the composite having the silver carbonate salt exhibits the best antimicrobial properties after 7 days. It is believed that this is due to the combination of the release profile and form of soluble silver released particular to this composite.

Unlike many other physical forms of silver salt, the nanoparticles of silver salt present in the composite material are non-toxic, non-allergenic, not photosensitive, do not degrade over time, and do not stain the skin.

Composites containing silver salt nanoparticles of different weight percents (6.21, 3.57, 1.34, 0.67, 0.37, 0.15 wt %) prepared according to the fourth aspect of the invention were found to exhibit pure Ag₂CO₃ at all concentrations or loadings of the silver salt. There is a greater affinity to form the carbonate salt on the aluminium oxide mesoporous substrate as no compositional change of the salt formed was observed at any concentration. Composites having a low silver salt content, small silver salt nanoparticles with a high dispersity over the substrate, create a bigger inhibition zone and kill a greater number of bacteria then composites having a higher silver salt content and larger nanoparticle size.

It will be appreciated that various embodiments and applications of the composite are envisaged and are not limited to the embodiments and applications hereinbefore described but may be varied in construction, detail and application within the scope of the appended claims.

1. A composite material comprising, a substrate and a silver salt.

2. A composite material as claimed in claim 1, wherein the substrate is amorphous or porous.

3. A composite material as claimed in claim 2, wherein the substrate is mesoporous.

4. A composite material as claimed in claim 3, wherein the mesoporous substrate has a pore size between 1 nm and 50 nm.

5. A composite material as claimed in claim 4, wherein the pore size is between 2 nm and 15 nm.

6. A composite material as claimed in claim 1, wherein the substrate comprises a metal oxide.

7. A composite material as claimed in claim 6, wherein the metal oxide is aluminium oxide.

8. A composite material as claimed in claim 1, wherein the silver salt is water soluable and/or biocompatible and/or thermally degradeable.

9. A composite material as claimed in claim 1, wherein the silver salt is silver carbonate.

10. A composite material as claimed in claim 1, wherein the silver salt is in the form of nanoparticles located at a surface of the substrate.

11. A composite material as claimed in claim 10, wherein the average diameter of the nanoparticles is between 1 nm and 9 nm.

12. A composite material as claimed in claim 11, wherein the average diameter of the nanoparticles is between 3 nm and 6 nm.

13. A composite material precursor comprising, by weight percent, an aluminium-alkoxide (5-50%), a silver salt (5-50%), a surfactant (0-25%), an alcohol (0-70%) and water (0-25%).

14. A composite material precursor as claimed in claim 13, wherein the aluminium-alkoxide is aluminium-sec-butoxide.

15. A composite material precursor as claimed in claim 13, wherein the silver salt is silver nitrate.

16. A method of forming a composite material from a composite material precursor comprising the steps of:
mixing, by weight percent, an aluminium-alkoxide (5-50%), a silver salt (5-50%), a surfactant (0-25%), an alcohol (0-70%) and water (0-25%) to form a solution;
heating the solution; and
allowing the solution to cool to form a solid composite material having a modified silver salt deposited on its surface.

17. A method of forming a composite material as claimed in claim 16, wherein the solution is heated to at least 500° C.

18. A method of forming a composite material as claimed in claim 16, wherein the solution is exposed to air or an atmosphere containing carbon dioxide.

19. A method of forming a composite material comprising the steps of:
providing a substrate;
coating the surface of the substrate with a layer comprising silver salt;
heating the substrate and layer of silver salt; and
allowing the substrate and layer of silver salt to cool to form a solid composite material having a modified silver salt deposited on the surface of the substrate.

20. A method of forming a composite material as claimed in claim 19, wherein the substrate and layer of silver salt is heated to at least 500° C.

21. A method of forming a composite material as claimed in claim 19, wherein the substrate and layer of silver salt is exposed to air or an atmosphere containing carbon dioxide.

22. A device comprising a composite material as claimed in claim 1.

23. A device as claimed in claim 22, wherein the device is a medical device.

24. A wound dressing comprising the composite material as claimed in claim 1

25. An antimicrobial agent comprising the composite material as claimed in claim 1.
